(19) United States
(12) Patent Application Publication
(10) Pub. No.: US 2006/0229630 A1
(43) Pub. Date: Oct. 12, 2006

(54) Surgical introducer

(76) Inventors: Simon Nicholas Collins, Tetbury (GB); Peter James Climer, Cirencester (GB)

Correspondence Address: YOUNG & THOMPSON, 745 SOUTH 23RD STREET, 2ND FLOOR, ARLINGTON, VA, 22202, US

(21) Appl. No.: 11/331,000

(22) Filed: Jan. 13, 2006

(30) Foreign Application Priority Data
Mar. 1, 2005 (GB) 0504123.1

(51) Int. Cl. A61F 2/00

(52) U.S. Cl. 606/91

(57) ABSTRACT

A head for a surgical introducing tool includes a cup holding element for releasably holding an acetabular cup, and a retaining element for preventing undesirable release of the acetabular cup from the holding element. The holding element is receivable in a space defined by an articulating inner surface of the acetabular cup and includes a seal and an air passage by which the acetabular cup can be held to the head by vacuum. The retaining element is positioned radially outwardly relative to the holding element and dimensioned to be a close fit with the acetabular cup. In use, relative tilting of polar axes of the head and the acetabular cup is prevented or limited by the retaining element while the cup is held by the holding element, so that accidental separation of the cup from the holding element is prevented.

### BACKGROUND OF THE INVENTION

The present invention relates to a surgical introducing tool for an acetabular cup, and to a head for the tool which can hold the acetabular cup.

It is known from U.S. Pat. No. 3,859,992 and FR2797180 to provide a surgical introducing tool for an acetabular cup which utilises vacuum to hold the cup to the tool.

However, a specific problem with these known arrangements, and also with most other kinds of acetabular cup introducing tools, is that a surgeon will typically tilt the tool in different directions, once the acetabular cup has been placed in the patient's acetabulum, to reposition the cup at the optimum angle. Especially with the introducers using vacuum to hold the cup to the tool, this tilting movement results in the cup breaking free from the tool due to the relative angular displacement of the longitudinal axis of the tool and the polar axis of the cup.

The present invention seeks to provide a solution to this problem.

### SUMMARY OF THE INVENTION

According to a first aspect of the present invention, there is provided a head for a surgical introducing tool, the head comprising: a cup holding element for releasably holding an acetabular cup, the holding element including a seal and an air passage by which the acetabular cup can be held to the head by vacuum; and characterised by a retaining element for preventing undesirable release of the acetabular cup from the holding element, the retaining element being positioned radially outwardly relative to the holding element and dimensioned to be a close fit with the acetabular cup, so that, in use, relative angular displacement of polar axes of the head and the acetabular cup is prevented or limited by the retaining element while the cup is held by the holding element, so as to prevent the cup from being accidentally released from the holding element.

According to a second aspect of the invention, there is provided a surgical introducing tool for an acetabular cup, the tool comprising a shaft having a handle at one end, and a head in accordance with the first aspect of the invention at the other end of the shaft.

According to a third aspect of the invention, there is provided an acetabular cup in combination with a head according to the first aspect of the invention, the cup having one or more formations adapted to accept the or each said retaining element of the head, wherein the or each formation is not an elongate slot having closed ends and being formed in a rim of the cup.

The invention will now be more particularly described, by way of example only, with reference to the accompanying drawings.

### BRIEF DESCRIPTION OF THE DRAWINGS

FIG. 1 is a diagrammatic elevational view of one embodiment of a surgical introducing tool for an acetabular cup, in accordance with the second aspect of the invention, and having a first embodiment of a head in accordance with the first aspect of the invention;

FIG. 2 is an enlarged view of the head shown in FIG. 1, and an acetabular cup;

FIG. 3 is a scrap pictorial view showing exaggerated relative angular displacement between the polar axis of the head and the polar axis of the cup, when engaged;

FIG. 4 is a diagrammatic plan view from above of a second embodiment of a head, in accordance with the first aspect of the invention;

FIG. 5 is a diagrammatic elevational view of a third embodiment of a head, in accordance with the first aspect of the invention;

FIG. 6 is a diagrammatic plan view from below of the head shown in FIG. 5;

FIG. 7 is a diagrammatic elevational view of a fourth embodiment of a head, in accordance with the first aspect of the invention, shown being offered towards an embodiment of an acetabular cup, in accordance with the third aspect of the invention;

FIG. 8 is a view similar to that shown in FIG. 7 with the head and acetabular cup engaged;

FIG. 9 is a diagrammatic elevational view of a fifth embodiment of a head, in accordance with the first aspect of the invention, shown being offered towards another embodiment of an acetabular cup, in accordance with the third aspect of the invention;

FIG. 10 is a view similar to that shown in FIG. 9 with the head and acetabular cup engaged;

FIG. 11 is a diagrammatic elevational view of a sixth embodiment of a head, in accordance with the first aspect of the invention, shown being offered towards a further embodiment of an acetabular cup, in accordance with the third aspect of the invention;

FIG. 12 is a view similar to that shown in FIG. 11 with the head and acetabular cup engaged;

FIG. 13 is a diagrammatic elevational view of a seventh embodiment of a head, in accordance with the first aspect of the invention, shown being offered towards an embodiment of an acetabular cup, in accordance with the third aspect of the invention; and

FIG. 14 is a view similar to that shown in FIG. 13 with the head and acetabular cup engaged.

### DESCRIPTION OF THE PREFERRED EMBODIMENTS

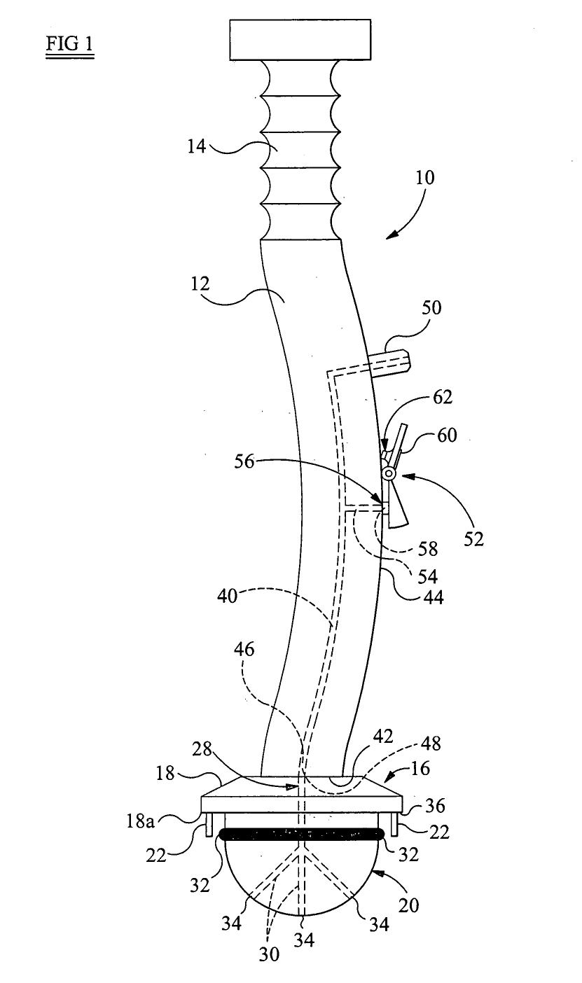

Referring firstly to FIGS. 1 to 3, there is shown a surgical introducing tool 10 which comprises a shaft 12 having a handle 14 at one end and a head 16 at the other end.

The shaft 12, in this case, is non-rectilinear, being cranked to promote simplified access to a patient's acetabulum during use, particularly during minimal access surgery. However, the shaft 12 may be rectilinear.

The head 16 includes a base plate 18, cup holding element 20 provided centrally on one major surface 18a of the base plate 18, and a plurality of retaining elements 22.

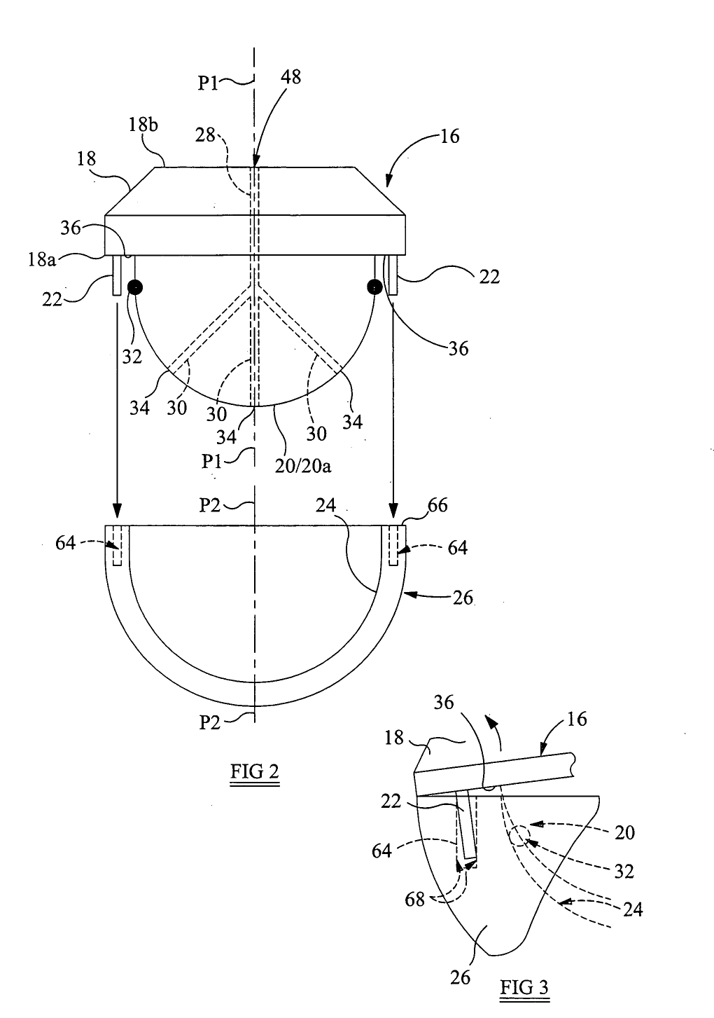

The cup holding element 20 is dimensioned to be a complementary or substantially complementary fit with an articulating inner surface 24 of a known acetabular cup 26, as can be understood from FIG. 2. A head air passage 28 passes centrally through the base plate 18, from the other major surface 18b, into the cup holding element 20. The head air passage 28, in the cup holding element 20, branches into three sub-passages 30 which open out on the surface 20a of the cup holding element 20, at positions which are spaced from each other and which are generally remote from the base plate 18.

The cup holding element 20 also includes an O-ring seal 32 which extends around the cup holding element 20 at a position adjacent to the equatorial diameter of the cup 26. The O-ring seal 32 is thus located between the base plate 18 and openings 34 of the air sub-passages 30.

The base plate 18 is dimensioned to overlap the cup holding element 20, thereby providing a flange 36 which extends around the cup holding element 20. The retaining elements 22 project from the flange 36, in spaced relationship to both the cup holding element 20 and each other. In this embodiment, three retaining elements 22 are provided (only two being shown in FIGS. 1 and 2), and are spaced equiangularly around the cup holding element 20.

The retaining elements 22 project from the flange 36 in parallel with each other, and extend arcuately along the surface of the flange 36.

The head 16 is screwthreadingly engagable, or otherwise modularly connectable, at the base plate 18 with the end of the shaft 12 opposite the handle 14. This allows pre-operative and intra-operative changing of the head 16 to one of different dimensions, for example when a different size of acetabular cup is required. However, the head may be permanently connected to the shaft.

The shaft 12 includes an internal air passage 40 which extends partway along the longitudinal extent of the shaft 12. The shaft air passage 40 opens out at the end 42 of the shaft 12 opposite the handle 14, and exits through a side 44 of the shaft 12 partway between the ends. Opening 46 of the shaft air passage 40 in the end 42 of the shaft 12 is positioned to align with opening 48 of the head air passage 28 on the base plate 18 of the head 16. One or more seals (not shown) are provided which, when the head 16 is engaged with the shaft 12, enables the head- and shaft-air passages 28 and 40 to fluid-tightly communicate and thus form a continuous air passage through the tool 10.

The exit of the shaft air passage 40 through the side 44 of the shaft 12 is via a nipple connector 50 integrally formed on the shaft 12. The nipple connector 50 enables connection of a suction line from remote air suction device (not shown).

The shaft 12 also includes a vent arrangement 52 for the shaft air passage 40. In this embodiment, the vent arrangement 52 comprises a branch passage 54 which leads from the shaft air passage 40 and opens out on the side 44 of the shaft 12, and an air-tight cap 56 or stop which covers an opening 58 on the shaft 12 to the branch passage 54. The air-tight cap 56 is provided on a pivotable lever 60 which can be operated via a user's thumb, for example. The lever 60 is urged, typically by a spring 62, to assume a condition whereby the air-tight cap 56 normally seals the opening 58 to the branch passage 54.

In use, and once the head 16 is attached to the shaft 12, an acetabular cup 26 of suitable size is urged onto the cup holding element 20 and retaining elements 22 until rim 66 abuts the base plate 18. The cup holding element 20 is dimensioned so that the O-ring seal 32 forms a fluid-tight interference fit on the articulating inner surface 24 of the acetabular cup 26, and the retaining elements 22 are received as close or tolerance fits in respective openings or slots 64 in the rim 66 of the acetabular cup 26. See FIG. 2.

An air line of an air suction device, which is usually available in theatre, is connected to the nipple connector 50 and a vacuum is generated in the space between the articulating inner surface 24 of the acetabular cup 26, the cup holding element 20, and the O-ring seal 32. The acetabular cup 26 is thus securely held by the introducing tool 10.

The acetabular cup 26 is introduced into the patient's body and positioned by impaction in the acetabulum. Once located, the surgeon can move the handle 14 of the tool 10 in a reciprocating manner, in other words angularly displace the longitudinal axis of the tool, to position the cup 26 at the optimum angle. During this tilting movement, and since the acetabular cup 26 will now be held securely in the patient's acetabulum, slight relative angular displacement of polar axis P1 of the head 16 and polar axis P2 of the cup 26 occurs. This results in slight separation or tilting of the head 16/base plate 18 from the cup 26. The retaining elements 22 of the head 16 consequently move in the openings or slots 64 in the rim 66 of the acetabular cup 26. See FIG. 3. Each retaining element 22, when titled due to the slight separation, will tend to bind on the interior surfaces 68 of its respective rim slot 64, thus preventing excessive movement which would otherwise cause the fluid-tight seal generated by the O-ring seal 32 to break. Breakage of the fluid-tight seal causes a loss of vacuum and the acetabular cup 26 to be no longer securely gripped by the tool 10.

Once the surgeon is satisfied that the acetabular cup 26 is securely located in the patient's acetabulum, the pivotable lever 60 on the shaft 12 is operated to lift the air-tight cap 56 and release the vacuum between the cup holding element 20 and the articulating inner surface 24 of the cup 26. The tool 10 can then be easily removed from the cup 26.

Obviously, the closer the tolerance of the fit between the retaining elements 22 and the respective slots 64 in the rim 66 of the acetabular cup 26, the less relative movement impartable therebetween, and the less relative angular displacement between the longitudinal axis of the tool 10/polar axis P1 of the head 16 and the polar axis P2 of the cup 26.

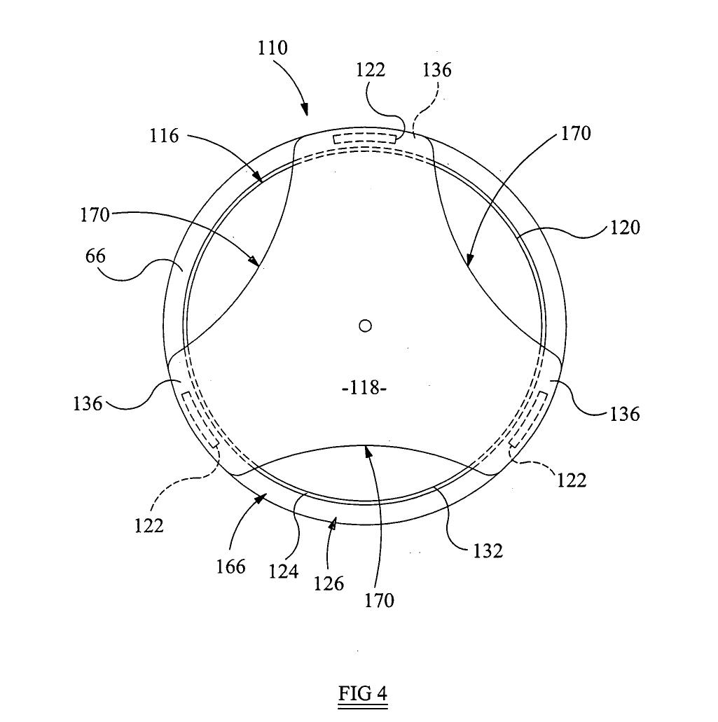

FIG. 4 shows a second embodiment of a head 116 of the present invention. Similar references refer to similar parts, and further detailed description is omitted.

The head 116 includes a base plate 118, cup holding element 120, and retaining elements 122, as in the first embodiment. The base plate 118, however, is non-circular, having equi-angularly spaced radially inwardly projecting recesses 170 around the circumference. The number of recesses 170 corresponds to the number of retaining elements 122. In this case, three recesses 170 are provided, which produces three flanges 136 from which the retaining elements 122 extend.

The cup holding element 120 is not correspondingly recessed, due to an interference equatorial or substantially equatorial fit being required between an O-ring seal 132 of the cup holding element 120 and an articulating inner surface 124 of an acetabular cup 126.

The recesses 170 in the base plate 118 allows a user of a tool 110 to see at least a rim 166 of the acetabular cup 126. This is particularly beneficial during introduction and location of the cup 126 into a patient's acetabulum.

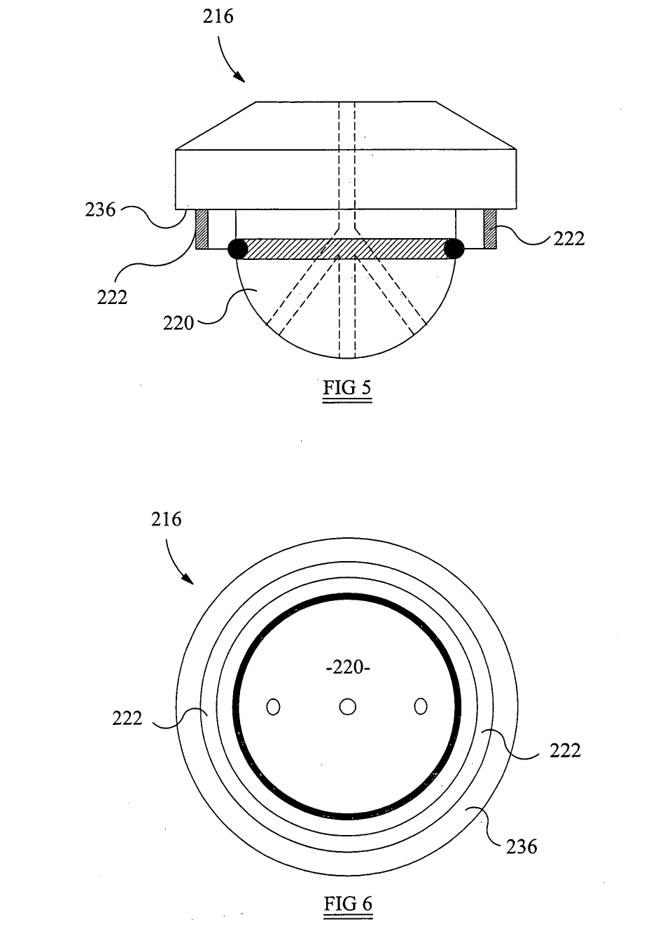

FIGS. 5 and 6 show a third embodiment of a head 216 of the present invention. Similar references refer to similar parts, and further detailed description is omitted.

In this embodiment, only a single retaining element 222 is provided on a flange 236 of head 216. The retaining element 222 is spaced radially outwards from a cup holding element 220, and extends continuously in a circumferential direction to surround the cup holding element 220.

An acetabular cup (not shown) has a complementarily or substantially complementarily shaped continuous opening, being a slot or channel, formed in a rim of the cup, a continuous inner shoulder formed adjacent to the rim, or a continuous outer shoulder formed adjacent to the rim, depending on the spacing of the retaining element 222 from the cup holding element 220.

In a modification, the retaining element 222 could be a continuous opening, in other words a slot or channel, formed in flange 236 of the head 216, and the acetabular cup therefore has a complementarily or substantially complementarily shaped projection positioned as described above.

In a further modification, two radially spaced concentric continuous retaining elements could be utilised. Complementarily or substantially complementarily shaped formations, being openings or projections, adapted to accept the retaining elements are therefore provided on the acetabular cup.

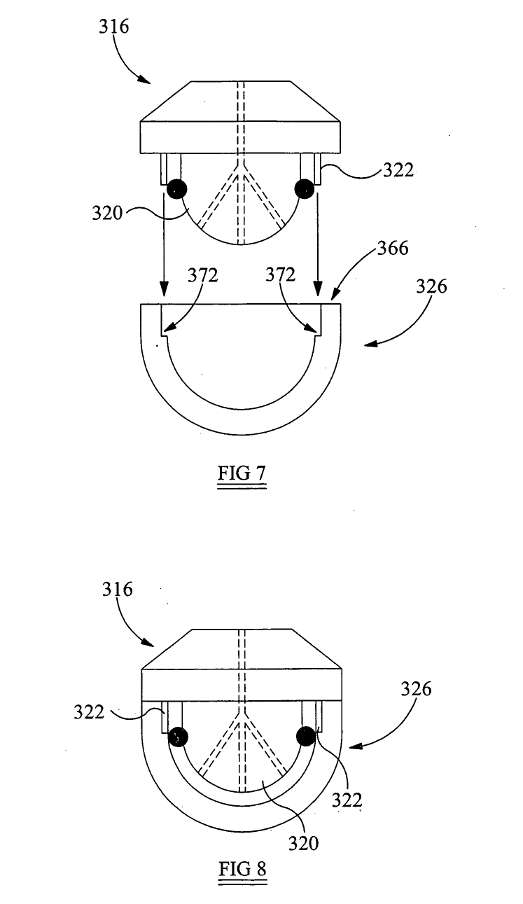

FIGS. 7 and 8 show a fourth embodiment of a head 316 and an embodiment of an acetabular cup 326. Similar references refer to similar parts, and further detailed description is omitted.

In this embodiment, the acetabular cup 326 includes one or more radially inner shoulders 372 adjacent to rim 366. In use, one or more retaining elements 322 is/are suitably spaced from cup holding element 320 to closely fit the or each shoulder 372.

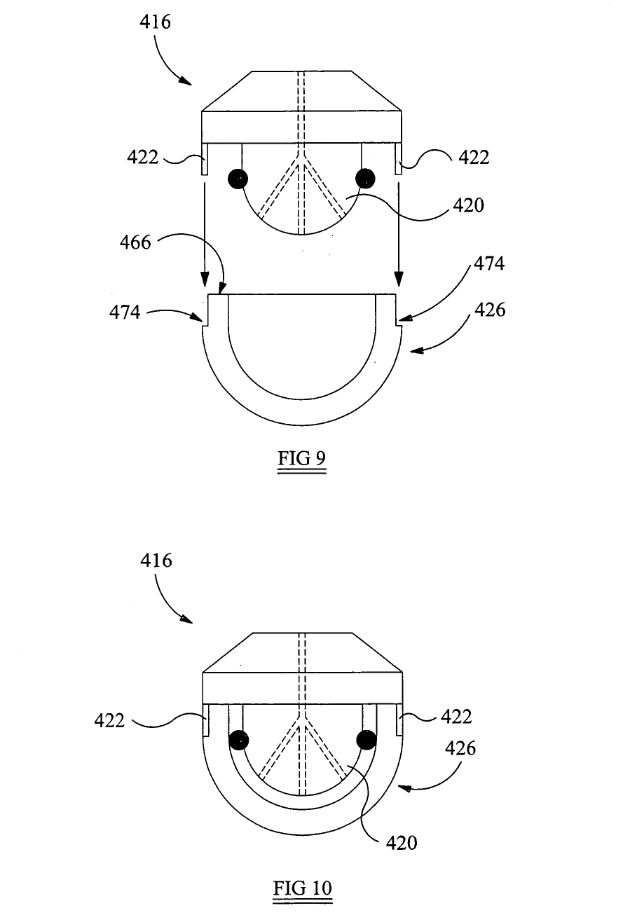

FIGS. 9 and 10 show a fifth embodiment of a head 416 and another embodiment of an acetabular cup 426. Similar references refer to similar parts, and further detailed description is omitted.

In this embodiment, the acetabular cup 426 includes one or more radially outer shoulders 474 adjacent to rim 466. In use, one or more retaining elements 422 is/are suitably spaced from cup holding element 420 to closely fit the or each outer shoulder 474.

In the fourth and fifth embodiments, it is envisaged that the acetabular cup can, additionally or alternatively, include one or more projections at or adjacent to the inner edge of the rim or the outer edge of the rim, and the or each respective retaining element of the head would therefore be an opening.

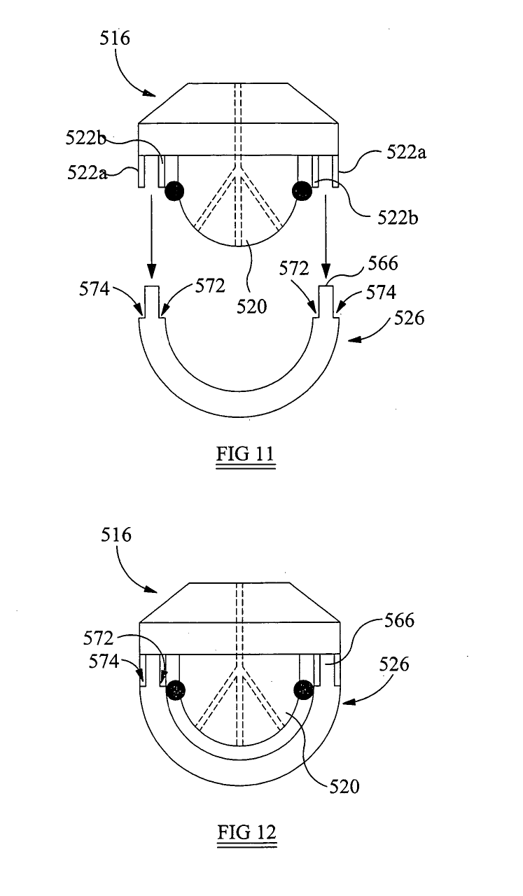

FIGS. 11 and 12 show a sixth embodiment of a head 516, and another embodiment of an acetabular cup 526. Similar references refer to similar parts, and further detailed description is omitted.

The acetabular cup 526 includes radially inner and outer shoulders 572 and 574 adjacent to rim 566. In use, retaining elements 522a and 522b are suitably spaced from cup holding element 520 to closely fit the shoulders 572 and 574. Retaining element 522a is spaced radially outwardly relative to the retaining element 522b.

Similarly to the earlier embodiments, in a modification, one or more projections could, additionally or alternatively, be included at or adjacent to inner and outer edges of the rim of the acetabular cup, and the respective retaining elements of the head would therefore be openings.

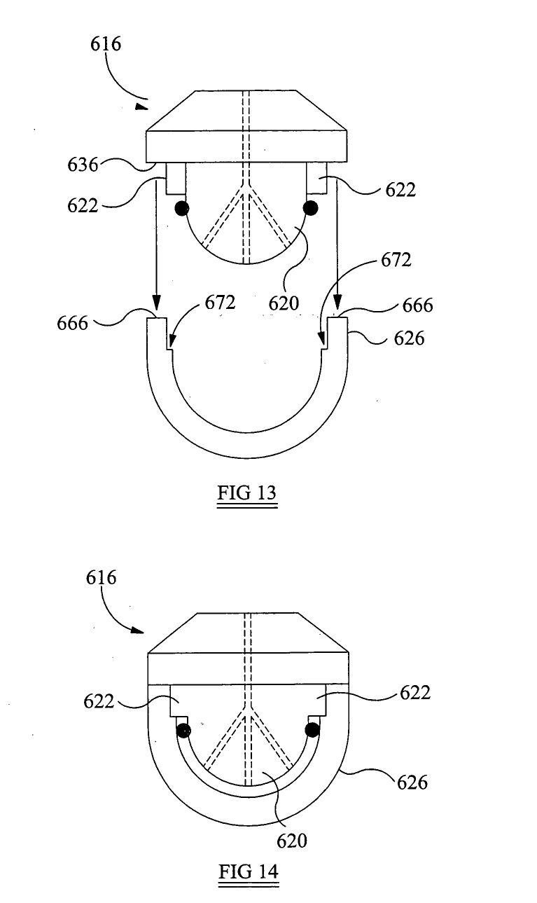

FIGS. 13 and 14 show a seventh embodiment of a head 616. Similar references refer to similar parts, and further detailed description is omitted.

In this embodiment, one or more retaining elements 622 is/are integrally formed with cup holding element 620 and extend from flange 636. Acetabular cup 626 includes one or more corresponding radially inner shoulders 672 adjacent to rim 666. In use, the or each retaining element 622 radially projects from cup holding element 620 to closely fit the or each inner shoulder 672.

It is envisaged that, in a modification to this embodiment, one or more of the retaining elements could be spaced from the flange of the head.

Except in the case of a continuous retaining element, the retaining elements described above also prevent relative rotational displacement of the head and the cup.

Any suitable number of retaining elements, and therefore corresponding recesses, can be provided. Thus, only a single retaining element may be required.

Although the retaining elements are described as being tongues, the retaining elements can be pins or any other suitably shaped projection. The or each opening, which includes a shoulder and a recess, when in the acetabular cup may be a slot, channel, or any close- or tolerance-fitting complementarily shaped opening.

As mentioned, it is possible that the rim of the acetabular cup can include one or more projections, instead of, or additionally to, the openings or slots. In this case, the retaining elements will be, or include, openings or slots formed in the flange or flanges of the base plate. These corresponding openings or slots will be dimensioned to closely fit the or each projection on the cup.

The retaining element or elements of the head have a close or tolerance fit with the or each corresponding formation on or adjacent to the rim of the cup, in at least a radial direction of the cup holding element.

The cup holding element described above can be any shape providing an interference fit is provided to enable formation of the vacuum seal by the O-ring seal. Regardless of shape, the cup holding element will still inherently have a polar axis.

Although three air sub-passages are suggested in the cup holding element, any suitable number of passages, including a single sub-passage, can be utilised.

Any suitable vent arrangement can be provided.

It is thus possible to provide a head for a surgical introducing tool that more securely holds an acetabular cup during insertion. It is also possible to provide a head for a surgical introducing tool which prevents or limits relative angular displacement of the longitudinal axis of the tool and the polar axis of the acetabular cup.

The embodiments described above are given by way of examples only, and modifications will be apparent to persons skilled in the art without departing from the scope of the invention as defined by the appended claims.

What is claimed is:

1. A head for a surgical introducing tool, the head comprising:
a cup holding element for releasably holding an acetabular cup, the holding element including a seal and an air passage by which the acetabular cup can be held to the head by vacuum; and
a retaining element for preventing undesirable release of the acetabular cup from the holding element, the retaining element being positioned radially outwardly relative to the holding element and dimensioned to be a close fit with the acetabular cup,
so that, in use, relative angular displacement of polar axes of the head and the acetabular cup is prevented or limited by the retaining element while the cup is held by the holding element, so as to prevent the cup from being accidentally released from the holding element.

2. A head as claimed in claim 1, wherein the head includes a flange on or in which the retaining element is positioned in spaced relationship to the holding element.

3. A head as claimed in claim 1, wherein the head includes a flange from which the retaining element extends, and the retaining element is integrally formed with the holding element.

4. A head as claimed in claim 2, wherein the flange surrounds the holding element, and a plurality of said retaining elements are positioned on or in the flange.

5. A head as claimed in claim 2, wherein the flange surrounds the holding element, and a single continuous retaining element is positioned on or in the flange.

6. A head as claimed in claim 2, wherein a plurality of flanges are provided angularly spaced around the holding element, a said retaining element being positioned on or in each flange.

7. A head as claimed in claim 1, wherein a further retaining element is positioned radially outwardly relative to the first said retaining element.

8. A head as claimed in claim 1, wherein the head includes one or more recesses which project radially inwards.

9. A head as claimed in claim 1, wherein the holding element is dimensioned to be an interference fit in at least a portion of the space defined by the articulating inner surface of the acetabular cup.

10. A head as claimed in claim 1, wherein the, each or at least one retaining element is a projection or an opening.

11. A surgical introducing tool for an acetabular cup, the tool comprising a shaft having a handle at one end, and a head as claimed in claim 1 at the other end of the shaft.

12. A tool as claimed in claim 11, wherein the head is releasably engaged with the said other end of the shaft.

13. A tool as claimed in claim 11, wherein an air passage is formed in the shaft, one end of the air passage fluidly communicating with the air passage in the head, and the other end being connectable to an air suction device.

14. A tool as claimed in claim 13, further comprising a vent arrangement for admitting air to the shaft air passage.

15. An acetabular cup in combination with a head as claimed in claim 1, the cup having one or more formations adapted to accept the or each said retaining element of the head, wherein the or each formation is not an elongate slot having closed ends and being formed in a rim of the cup.

16. An acetabular cup as claimed in claim 15, wherein the or each formation is an opening formed in a rim of the acetabular cup, a radially inner shoulder formed adjacent to the rim of the acetabular cup, and/or a radially outer shoulder formed adjacent to the rim of the acetabular cup.

17. An acetabular cup as claimed in claim 16, wherein a plurality of rim openings, inner shoulders and/or outer shoulders are provided.

18. An acetabular cup as claimed in claim 16, wherein a single rim opening, inner shoulder and/or outer shoulder is provided.

19. An acetabular cup as claimed in claim 18, wherein the single rim opening, inner shoulder, and/or outer shoulder is continuous.

20. An acetabular cup as claimed in claim 15, wherein the, each or at least one formation is a projection formed on the rim of the acetabular cup, the projection being provided on or adjacent to an inner edge of the rim, on or adjacent to an outer edge of the rim, and/or partway between inner and outer edges of the rim.
